(19) United States
(12) Patent Application Publication
(10) Pub. No.: US 2009/0177088 A1
(43) Pub. Date: Jul. 9, 2009

(54) Ultrasonic Probe

(76) Inventors: Yasunobu Hasegawa, Saitama (JP); Kunihisa Taki, Saitama (JP)

Correspondence Address: EDWARDS ANGELL PALMER & DODGE LLP, P.O. BOX 55874, BOSTON, MA, 02205, US

(21) Appl. No.: 12/225,965

(22) PCT Filed: Feb. 16, 2007
(86) PCT No.: PCT/JP2007/053332

§ 371 Date: Oct. 2, 2008

(30) Foreign Application Priority Data
Aug. 31, 2006 (JP) 200723611
Jan. 6, 2007 (JP) 2007 000855

(51) Int. Cl. A61B 8/13

(52) U.S. Cl. 600/445

(57) ABSTRACT

A short axis scanning type ultrasonic wave probe configured such that: a plurality of strip shaped piezoelectric elements is arranged in a long axis direction, which is a crosswise direction of the piezoelectric elements, so as to form a flat piezoelectric element group; the piezoelectric element group is housed within a sealed container filled with a liquid that functions as an ultrasonic wave medium; and the piezoelectric element group is mechanically scanned in a short axis direction, which is a lengthwise direction of the piezoelectric elements, and the piezoelectric element group is linearly moved in the short axis direction so as to be mechanically scanned. Thereby, there is provided a short axis mechanical scanning probe in which the ultrasonic wave transmitting and receiving surface thereof can be easily brought into contact with a protruding section (such as a breast) of a subject human body, while realizing excellent lateral resolution.

### TECHNICAL FIELD

The present invention relates to an ultrasonic probe in which a piezoelectric element group is mechanically scanned in the short axis direction (hereinafter, referred to as “short axis mechanical scanning probe”), in particular, to a short axis mechanical scanning probe in which the piezoelectric element group is linearly moved in the short axis direction.

### BACKGROUND ART

### Background of the Invention

A short axis mechanical scanning probe, for example, performs long axis direction electronic scanning and short axis direction mechanical scanning (oscillation) on a piezoelectric element group to obtain a three dimensional image (Japanese Examined Patent Publication No. Hei 7-38851, Japanese Unexamined Patent Publication No. 2003-175033, and Japanese Patent Application No. 2005-175700 (unpublished reference)).

Such a probe has been brought to practical application because for example wiring (electrical connection) and scanning circuits thereof can be made simpler, compared for example to a matrix type in which piezoelectric elements are arranged in lengthwise and crosswise arrays to be electronically scanned in the two dimensional direction.

(Prior Art) FIG. 5 is a drawing for explaining a conventional example of a short axis mechanical scanning probe, wherein FIG. 5A is a sectional view in the long axis direction, and FIG. 5B is a sectional view in the short axis direction (along the arrow V-V).

As shown in FIG. 5, the short axis mechanical scanning probe is such that a piezoelectric element group 102 (ultrasonic wave frequency at 3 MHz for example) provided on a rotational retention base 101 is housed within a sealed container 103. The rotational retention base 101 is of a sectionally channel shape with leg sections 101a and 101b on both end sides of a horizontal section thereof, and on the horizontal section there is provided the piezoelectric element group 102. Moreover, on the inner side face of the one leg section 101b, there is fixed a first bevel gear 104a.

The piezoelectric element group 102 is formed such that a large number of piezoelectric elements 102a are arranged in the long axis direction (crosswise direction of the piezoelectric elements 102a), and it is fastened onto a backing member 105a on a curve-surfaced base 105 provided on the horizontal section of the rotational retention base 101. As a result, the ultrasonic probe is a so called convex type ultrasonic probe. On the surface of the piezoelectric element group 102, there is provided an acoustic matching layer 106a that brings the acoustic impedance close to that of a human body to increase propagation efficiency, and on the acoustic matching layer 106a there is further provided an acoustic lens 106. The respective piezoelectric elements 102a of the piezoelectric element group 102 are led out so as to be electrically connected to a flexible substrate (not shown in the drawing).

The sealed container 103 is integrated by fitting to each other, a container main body 103a and a cover 103b, the cross sections of which are both concave shaped. On a pair of opposing side walls of the container main body 103a, there is provided rotational center shafts 107 that rotate and oscillate the rotational retention base 101 in the short axis direction (lengthwise direction of the piezoelectric element 102a), and the rotational center shafts 107 slidably engage with bearings 107a and 107b of the leg sections 101a and 101b on both of end sides of the rotational retention base 101. At a bottom wall 103a of the container main body, there is provided a rotation shaft 108 connected to a forward and reverse rotating mechanism such as motor, and a second bevel gear 104b that passes in a sealed condition through the bottom wall 103a so as to mesh with the first bevel gear 104a. The rotation shaft 108 is supported on a rotation shaft bearing 115.

The inside of the sealed container 103 is filled with a liquid that serves as an ultrasonic wave medium such as oil L that results in bringing acoustic impedance close to that of a human body and with a low ultrasonic wave propagation loss. The oil L is filled into the sealed container 103 from an inlet hole (not shown in the drawing). Accordingly, ultrasonic wave propagation loss between the inner circumferential surface of the cover 103b and the piezoelectric element group 102 (acoustic lens 106) becomes lower, and the matching of the acoustic impedance with a human body is increased. As a result, ultrasonic wave propagation efficiency is increased. If air is present between the inner circumferential surface of the cover 103b and the surface of the piezoelectric element group 102, attenuation of the ultrasonic waves becomes significant and propagation efficiency becomes degraded. As a result, it is not possible to perform excellent transmission and reception of ultrasonic waves.

The rotating mechanism such as motor is covered by a back face cover 103c, and a coaxial cable connected to the flexible substrate is led out from this back face cover 120, and the coaxial cable is connected to a diagnostic tool. As a result, forward and reverse rotation of the second bevel gear 104b rotates and oscillates the first bevel gear 104a, and the rotational retention base 101 integrated with this rotates and oscillates left and right about the center line that equally divides the short axis direction of the piezoelectric element group 102.

### Problems in the Prior Art

However, in the conventional short axis mechanical scanning probe configured as described above, the piezoelectric element group 102 is electronic linear scanned in an arc shape in the short axis direction. Therefore, the transmitting and receiving surface of the sealed container 103 is also of a convex shape section of an arc shape in the short axis direction. Moreover, in this conventional example, the piezoelectric element group 102 is of a convex shape (convex shaped curved surface) in the long axis direction. Therefore the shape of the sealed container 103 in the long axis direction is also of a convex shape. Consequently, the transmitting and receiving surface in both of the short axis and long axis directions is of a convex shape, forming an overall convex shape (protruding shape).

As a result, there has been a problem in that it is difficult to bring the entire transmitting/receiving surface into contact with a breast (convex section, protruding section) in the case of diagnosing a mammary gland of for example a human body (female in particular). In the case where the entire transmitting and receiving surface is not in contact with the breast, attenuation of the ultrasonic waves occurs, making it impossible to obtain a normal diagnostic image of the subject human body.

Furthermore, since the conventional short axis mechanical scanning probe performs scanning in an arc shape in the short axis direction (lengthwise direction of the piezoelectric elements), there has been a problem in that the lateral resolution becomes rougher for a deeper section in a subject human body. In this case, the rotation (oscillation) speed of the piezoelectric element group 102 may be lowered. However, over time, this would cause a positional displacement resulting in a blur in the image. Therefore, it is better to have a high rotation speed.

These problems are observed not only in the case where the piezoelectric element group 102 is arranged in a convex shape, and similar problems occur in the case where the piezoelectric element group 102 is arranged on a flat surface.

Therefore, an object of the present invention is to provide a short axis mechanical scanning probe that can be easily brought into contact with a protruding section of a subject human body and that enables excellent lateral resolution.

### BRIEF DESCRIPTION OF THE DRAWINGS

FIG. 1 is a drawing for explaining a first embodiment of a short axis mechanical scanning probe of the present invention, wherein FIG. 1A is a sectional view in the long axis direction, and FIG. 1B is a sectional view in the short axis direction (sectional view taken along the arrow I-I in FIG. 1A).

FIG. 2 is a sectional view in the short axis direction for explaining operations (effects) of the first embodiment of the short axis mechanical scanning probe of the present invention.

FIG. 3 is a schematic drawing in the long axis direction for explaining operations of the first embodiment of the short axis mechanical scanning probe of the present invention, wherein FIG. 3A is a schematic view of a pulse application to the first five piezoelectric elements via a delay circuit, and FIG. 3B is a schematic view of a pulse application to the next switched five piezoelectric elements.

FIG. 4 is a sectional view in the long axis direction of a second embodiment of the short axis mechanical scanning probe of the present invention, showing an example of a first piezoelectric element group and a second piezoelectric element group arranged in parallel in the long axis direction.

FIG. 5 is a drawing for explaining a conventional example of a short axis mechanical scanning probe, wherein FIG. 5A is a sectional view in the long axis direction, and FIG. 5B is a sectional view in the short axis direction (sectional view taken along the arrow V-V in FIG. 5A).

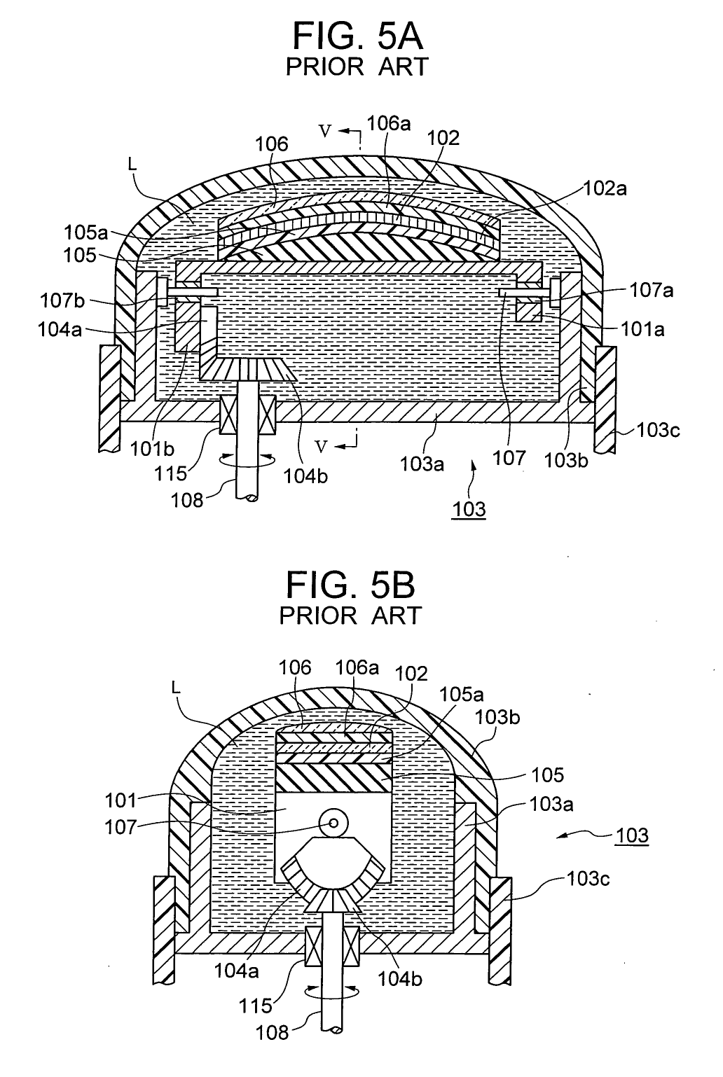

### DISCLOSURE OF THE INVENTION

The present invention configures a short axis scanning type ultrasonic wave probe such that: a plurality of strip shaped piezoelectric elements is arranged in a long axis direction, which is a crosswise direction of the piezoelectric elements, so as to form a flat piezoelectric element group; the piezoelectric element group is housed within a sealed container filled with a liquid that functions as an ultrasonic wave medium; and the piezoelectric element group is mechanically scanned in a short axis direction, which is a lengthwise direction of the piezoelectric elements, and the piezoelectric element group is linearly moved in the short axis direction so as to be mechanically scanned.

According to such a configuration, the piezoelectric element group linearly moves (reciprocates) in the short axis direction rather than rotating and oscillating in an arc shape in the short axis direction. Consequently, the transmitting and receiving surface of the sealed container does not need to be made in a convex shape as practiced in the conventional example, and it can be made in a flat surface. As a result, it becomes easier to bring the entire transmitting and receiving surface of the sealed container into contact with a subject human body such as a breast.

Furthermore, since the piezoelectric element group linearly moves (reciprocates) in the short axis direction, ultrasonic waves from the transmitting and receiving surface are emitted in parallel with a section to be examined. Consequently, intervals of ultrasonic waves are constant even in a deep section of a subject human body, thereby realizing excellent lateral resolution while increasing the movement speed of the piezoelectric element group.

Moreover, the present invention is configured such that: the piezoelectric element group is provided on a movable base; both end sides in the long axis direction of the movable base have a pair of leg sections, and guiding shafts are inserted in the short axis direction into the pair of leg sections; on one of the pair of leg sections there is fixed a movable rack; and a rotating gear (pinion) that uses a motor as its driving source meshes with the movable rack. Furthermore in the pair of leg sections in the long axis direction of the movable base, on which the piezoelectric element group is provided, there are provided guiding shafts inserted in the short axis direction. Consequently, the piezoelectric element group can be freely moved in the short axis direction. Here, the movable rack provided in the short axis direction on one of the leg sections of the movable base is moved by the rotating gear that uses the motor as a driving source.

Furthermore, in the present invention, the piezoelectric element group comprises a first piezoelectric element group and a second piezoelectric element group having different ultrasonic wave frequencies, and the first piezoelectric element group and the second piezoelectric element group are arranged in parallel in the long axis direction. Thereby, the first piezoelectric element group and the second piezoelectric element group having different ultrasonic wave frequencies can be switched to be used on demand. Therefore, a deep section and a superficial section (in the vicinity of the surface of a subject human body) of a subject human body can be observed with the same short axis mechanical scanning probe, while reducing the amount of operations.

### BEST MODE FOR CARRYING OUT THE INVENTION

### First Embodiment

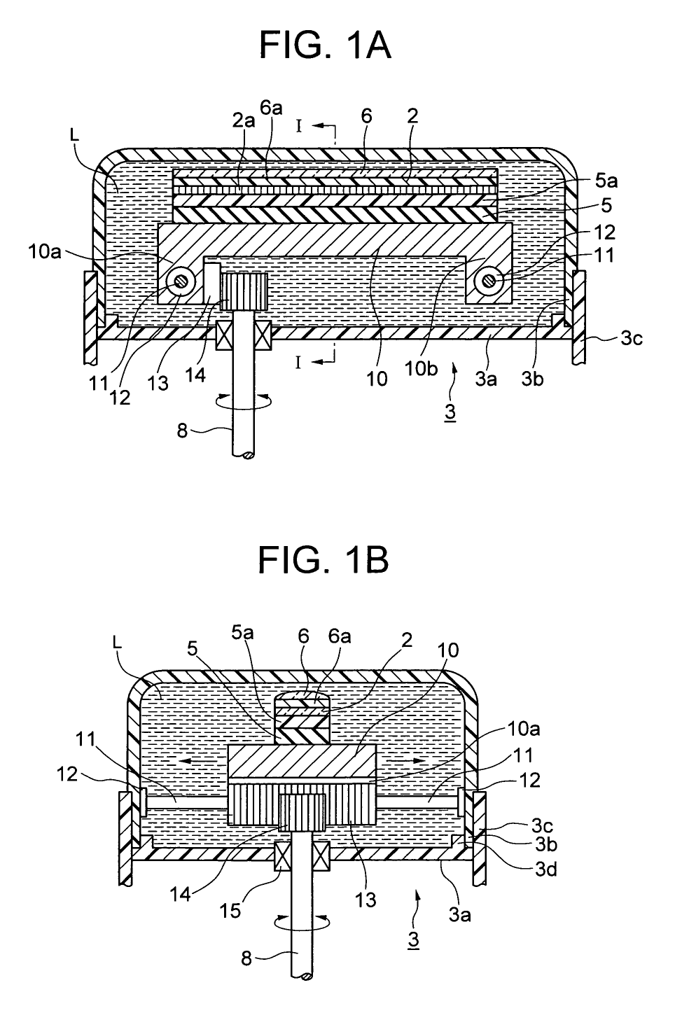

FIG. 1 is a drawing for explaining a first embodiment of a short axis mechanical scanning probe of the present invention, wherein FIG. 1A is a sectional view in the long axis direction, and FIG. 1B is a sectional view in the short axis direction (sectional view taken along the arrow I-I in FIG. 1A).

The short axis mechanical scanning probe of the present invention is such that a sealed container 3 filled with oil L that functions as an ultrasonic wave medium, houses a piezoelectric element group 2. The sealed container 3 is such that a container main body 3a having protrusions on the outer periphery thereof, engages with the inner periphery of a sectionally concave shaped cover 3b having a flat transmitting and receiving surface. The piezoelectric element group 2 is such that a plurality of piezoelectric elements 2a is arranged in the long axis direction, that is, the crosswise direction of the piezoelectric element group 2. Here, the piezoelectric elements 2a are arranged in a flat shape (on a plane face) rather than in a convex shape.

The piezoelectric element group 2 is fastened onto a backing member 5a of a flat plate shaped base 5, and the base 5 is fixed on a movable base 10. On the transmitting and receiving surface of the piezoelectric element group 2, there is provided an acoustic matching layer 6a, and there is further provided an acoustic lens 6 having a curvature (convex) in the short axis direction. The movable base 10 is of a sectionally channel shape and has a pair of leg sections 10a and 10b provided on both long axis direction end sides thereof. Through the pair of leg sections 10a and 10b there are inserted guiding shafts 11 in the short axis direction. Both of the ends of the guiding shaft 1 are, for example, fastened onto the inner circumferential surface in the short axis direction of the cover 3b by fastening devices 12. Here, the pair of protrusions 3d provided on the container main body 3a may be further extended upward so as to fix the guiding shafts 11 on the inner peripheries of the protrusion 3d.

On the inner side face of the one leg section 10a of the movable base 10, there is fixed a movable rack 13 that is straight in the short axis direction. The movable rack 13 meshes with a rotating gear 14 (pinion) and is freely movable in the short axis direction. The rotating gear 14 is provided on the tip end side of a rotation shaft 8, the driving source of which is a motor. As mentioned above, the rotation shaft 8 is journalled on a rotation shaft bearing 15 that is oil-sealed in the bottom wall of the container main body 3a.

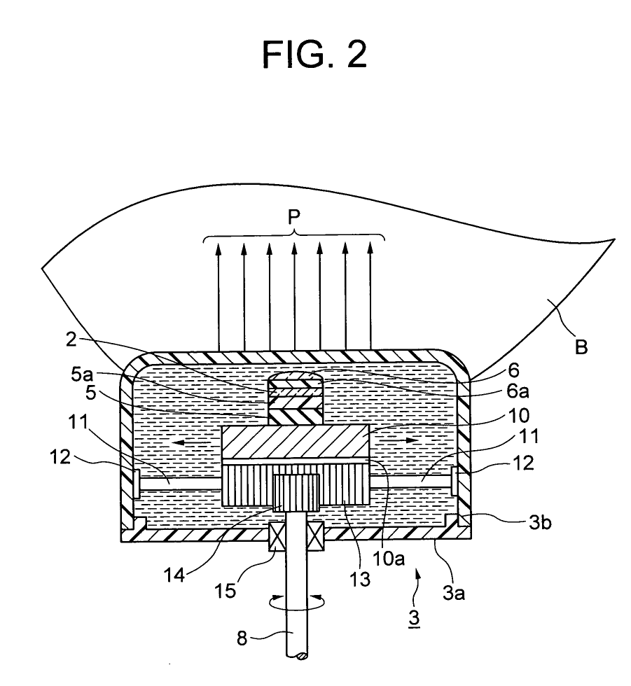

Thus, in the probe of the present invention, when the rotating gear 14 is rotated forward or reverse by a forward and reverse rotating motor serving as a driving source, the movable rack 13 is guided by the guiding shafts 11 so as to linearly move (reciprocate) in the short axis direction. Here, the movable rack 13 reciprocates (scans) between both end sides in the short axis direction with the initial position of the piezoelectric element group 2 taken as the center in the short axis direction. As a result, for example as shown in FIG. 2, in the short axis direction, ultrasonic waves P converged by the acoustic lens 6 are transmitted in parallel into a subject human body such as a breast B along with the movement of the piezoelectric element group 2.

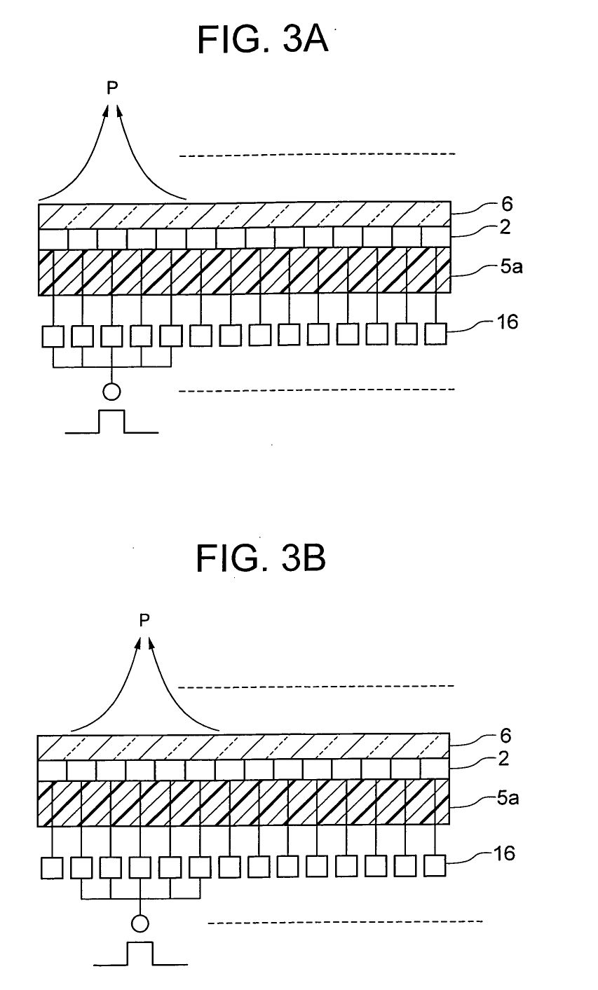

In the probe of the present invention, as shown for example in FIG. 3A and FIG. 3B, in the long axis direction, a pulse is applied via a delay circuit 16 to a plurality, for example five of the piezoelectric elements 2a from one end side of the piezoelectric element group 2 so as to electronically converge ultrasonic waves. Subsequently, the piezoelectric element group 2 is switched to the next five piezoelectric elements 2a and a similar pulse is applied to these piezoelectric elements 2a. This sequence is repeated, thereby sequentially converging ultrasonic waves to perform linear scanning in the long axis direction. By performing these scans, mechanical linear scanning is performed in the short axis direction, and electronic linear scanning is performed in the long axis direction in order to obtain a three dimensional image of the subject human body.

According to such a configuration, linear scanning is performed with the piezoelectric element group 2 that linearly moves in the short axis direction, and electronic linear scanning is performed in the long axis direction with the flat shaped transmitting and receiving surface. Consequently, both of the short axis direction and the long axis direction of the piezoelectric element group 2 are straight lines, and the transmitting and receiving surface of the sealed container 3 can be made into a flat surface. As a result, it becomes easier to bring the transmitting and receiving surface into close contact with a protruding section of a subject human body such as the breast B without having a gap therebetween (refer to FIG. 2). In general, when performing scanning, a gelatinous liquid agent that functions as an ultrasonic wave medium is applied in between the subjection human body and the transmitting/receiving surface.

Moreover, since mechanical linear scanning is performed in the short axis direction, ultrasonic waves P from the acoustic lens 6 are transmitted in parallel as shown in FIG. 2. Consequently, compared to the case of performing scanning in an arc shape (sector scanning), excellent lateral resolution can be achieved even when scanning a deep section of the subjection human body. Here, since electronic linear scanning is also performed in the long axis direction, excellent lateral resolution can be achieved in scanning in both of the short axis direction and the long axis direction.

The ultrasonic wave frequency used here is in a range between 3 MHz and 7.5 MHz as with the conventional example. As a result, the length of the piezoelectric element 2a in the short axis direction becomes half or less of that in the conventional example. In other words, at a higher ultrasonic wave frequency, the length of the piezoelectric element 2a becomes shorter. Accordingly, at a lower ultrasonic wave frequency, the ultrasonic waves are converged in a deep section of the subject human body, and at a higher ultrasonic wave frequency, the ultrasonic waves are converged in a more superficial section of the subject human body. Accordingly, piezoelectric elements with a lower ultrasonic wave frequency are appropriately used for a deeper section of a human body, and those with a higher ultrasonic frequency are appropriately used for in the vicinity of the surface of a human body.

### Second Embodiment

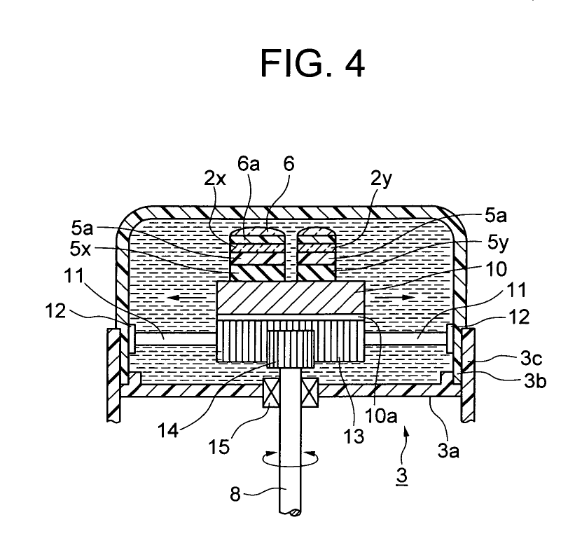

FIG. 4 is a sectional view in the short axis direction of a second embodiment of the short axis mechanical scanning probe of the present invention.

In this second embodiment, the above mentioned piezoelectric element group 2 comprises a first piezoelectric element group 2x and a second piezoelectric element group 2y provided parallel with each other in the long axis direction. Here the ultrasonic wave frequency of the first piezoelectric element group 2x and that of the second piezoelectric element group 2y are different. The ultrasonic wave frequency of the first piezoelectric element group 2x is 7.5 MHz and that of the second piezoelectric element group 2y is 10 MHz. Both of these are fastened onto backing members 5a on bases 5x and 5y, and the bases 5x and 5y are fixed on a movable base 10.

In such a probe, in the case of performing an examination on a deep section of a breast for example, the piezoelectric elements at a lower ultrasonic wave frequency (7.5 MHz) are used, and in the case of performing an examination on a superficial section in the vicinity of the surface of the subject human body, the piezoelectric elements at a higher ultrasonic wave frequency (10 MHz) are used. Here, a switching mechanism of an electric circuit (not shown in the drawing) switches so as to supply electrical pulses to either one of the first piezoelectric element group 2x and the second piezoelectric element group 2y.

Thereby, it is possible to appropriately switch for use, the first piezoelectric element group 2x and the second piezoelectric element group 2y respectively having different ultrasonic wave frequencies. Consequently, the amount of operations can be reduced compared to that with the probe of the first embodiment that requires the first and second piezoelectric element groups with different ultrasonic wave frequencies. Moreover, there is an advantage in that it is possible to examine and compare a deep section and a superficial section within a same region, while the short axis mechanical scanning probe of the second embodiment is in contact with a breast.

Furthermore, here the first piezoelectric element group 2x and the second piezoelectric element group 2y respectively having different ultrasonic wave frequencies are fastened onto the backing members 5a on the separate bases 5x and 5y. Consequently for example when forming a coating of an acoustic matching layer 6a on the piezoelectric element group 2, the operation thereof can be made easier. That is to say, since the first piezoelectric element group 2x and the second piezoelectric element 2y are respectively independent, the polishing operation for the piezoelectric elements with thicknesses corresponding to the respective ultrasonic wave frequencies can be made significantly easier.

### INDUSTRIAL APPLICABILITY

The short axis mechanical scanning probe of the present invention can be widely used for forming a three dimensional image of an examination subject such as a human body.

1. A short axis scanning type ultrasonic wave probe in which: a plurality of strip shaped piezoelectric elements is arranged in a long axis direction, which is a crosswise direction of said piezoelectric elements, so as to form a flat piezoelectric element group; said piezoelectric element group is housed within a sealed container filled with a liquid that functions as an ultrasonic wave medium; and said piezoelectric element group is mechanically scanned in a short axis direction, which is a lengthwise direction of said piezoelectric elements, wherein said piezoelectric element group is linearly moved in the short axis direction so as to mechanically scan.

2. A short axis scanning type ultrasonic wave probe according to claim 1, wherein: said piezoelectric element group is provided on a movable base positioned in said long axis direction; both end sides of said movable base have a pair of leg sections, and guiding shafts are inserted in said short axis direction into said pair of leg sections; on one of said pair of leg sections there is fixed a movable rack in said short axis direction; and a rotating gear drive-sourced by a motor meshes with said movable rack.

3. A short axis scanning type ultrasonic wave probe according to claim 1, wherein said piezoelectric element group comprises a first piezoelectric element group and a second piezoelectric element group having different ultrasonic wave frequencies, and said first piezoelectric element group and said second piezoelectric element group are arranged in parallel in the long axis direction.
